(19) United States
(12) Patent Application Publication
(10) Pub. No.: US 2007/0248943 A1
(43) Pub. Date: Oct. 25, 2007

(54) Displaying cellular analysis result data using a template

(75) Inventors: Ralph B. Taylor, Weston, FL (US); Ziling Huo, Miami, FL (US); Patricio J. Vidal, Miami, FL (US)

Correspondence Address: BECKMAN COULTER, INC., P.O. BOX 169015, MAIL CODE 32-A02, MIAMI, FL, 33116-9015, US

(73) Assignee: Beckman Coulter, Inc., Fullerton, CA (US)

(21) Appl. No.: 11/408,456

(22) Filed: Apr. 21, 2006

(51) Int. Cl. C12Q 1/00; G06F 19/00

(52) U.S. Cl. 435/4; 702/19

(57) ABSTRACT

In accordance with the principles of the invention, methods, systems, and computer-readable mediums are provided for displaying cellular analysis result data including accessing cellular analysis result data; determining frequency information of a plurality of cell populations from the cellular analysis result data; associating frequency information of each of the plurality of cell populations with a surface elevation point; and displaying an interactive surface plot of the associated frequency information and the surface elevation points.

### BACKGROUND OF THE INVENTION

1. Field of the Invention

The present invention relates generally to systems and methods of displaying data and, more specifically, relates to systems and methods for displaying cellular analysis result data.

2. Description of Related Art

Conventional cellular analyzers provide a user with the results of the cellular analysis by displaying the data in a two-dimensional histogram or scattergram representing a combination of two raw or transformed cell measurements. These histograms may be displayed using bitmaps to represent the frequency of each two-dimensional bin. Each bin location may be assigned a bitmap that corresponds to a frequency range. Physicians that visually inspect the displayed data need to be able to clearly identify and compare the frequency information of the different cell populations to help in the diagnosis of a patient and, in some cases, validate results.

FIG. 11 depicts details of an exemplary bitmap display where frequency ranges are represented as bitmaps of 4×4 pixels. Each different bitmap represents a different frequency range. The density of colors in the bitmaps is related to the frequency range. For low frequency ranges, few pixels in the bitmap are colored, and for high frequency ranges a high number of the pixels in the bitmap are colored. In addition, each cell population is identified by a family of color-related bitmaps (blue for reticulocytes, red for mature red blood cells, etc.).

Conventional methods of display include contour plots, bitmaps, and proportional-color plots. However, these methods have disadvantages as they fail to clearly show in detail the different frequency values and the relationship between the different cell populations. Contour plots and bitmaps only show ranges of frequencies. Further, contour and proportional-color plots cannot identify different cell populations by using different colors.

As such, there is a need for systems and methods that provide for displaying frequency information in relation to the different cell populations.

### SUMMARY OF THE INVENTION

In accordance with the principles of the present invention, methods, systems, and computer-readable mediums are provided for displaying cellular analysis result data including accessing cellular analysis result data; determining frequency information of a plurality of cell populations from the cellular analysis result data; associating frequency information of each of the plurality of cell populations with a surface elevation point; and displaying an interactive surface plot of the associated frequency information and the surface elevation points.

### BRIEF DESCRIPTION OF THE DRAWINGS

The patent or application file contains at least one drawing executed in color. Copies of the patent or patent application publication with color drawings will be provided by the Office upon request and payment of the necessary fee.

The accompanying drawings, which are incorporated in and constitute a part of this specification, illustrate several embodiments of the invention and together with the description, serve to explain the principles of the invention, and, together with the description, explain the features and aspects of the invention. In the drawings,

FIG. 1 is an exemplary diagram of an exemplary system environment in which systems and methods, consistent with the principles of some embodiments of the present invention, may be implemented;

FIG. 2 is an exemplary diagram of main components of a computer, consistent with some embodiments of the principles of the present invention;

FIG. 3 is an exemplary diagram of components of a server, consistent with the principles of some embodiments of the present invention;

FIG. 4 depicts an exemplary flow diagram of the steps performed by a computer consistent with the principles of some embodiments of the present invention;

FIG. 5 depicts an exemplary flow diagram of the steps performed by a computer consistent with the principles of some embodiments of the present invention;

FIG. 6 depicts an exemplary display provided to a user consistent with the principles of some embodiments of the present invention;

FIG. 7 depicts an exemplary display provided to a user consistent with the principles of some embodiments of the present invention;

FIG. 8 depicts an exemplary display provided to a user consistent with the principles of some embodiments of the present invention;

FIG. 9A-D depicts an exemplary display provided to a user consistent with the principles of some embodiments of the present invention;

FIG. 10 depicts an exemplary display provided to a user consistent with the principles of some embodiments of the present invention; and

FIG. 11 depicts an exemplary bitmap display.

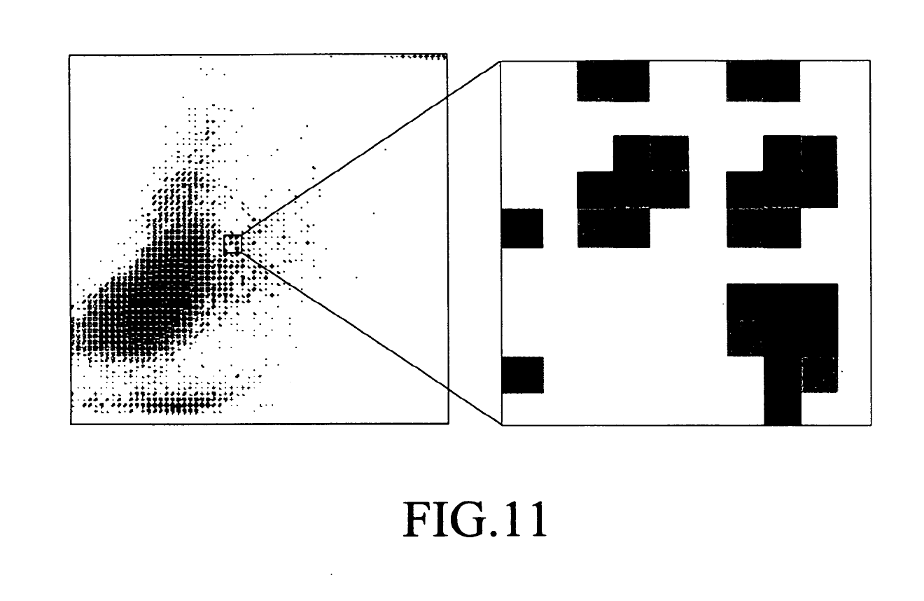

### DETAILED DESCRIPTION

Reference will now be made in detail to the present invention, examples of which are illustrated in the accompanying drawings. Wherever possible, the same reference numbers will be used throughout the drawings to refer to the same or like parts.

Overview

Methods and systems consistent with the principles of some embodiments of the present invention provide for a system that enables a user to view and/or analyze displayed data representing cellular analyzer test results. Specifically, methods and systems, consistent with some embodiments of the present invention provide for enabling a user to view two-dimensional histogram data from a cellular analysis as a surface plot, and to use color to identify cell populations. The surface plot may further provide interactivity and may further provide pre-defined views in order to allow more detailed inspection of the frequency distributions.

Cellular Analysis

The present invention may be used to analyze various types of cells, cellular components, body fluids and/or body fluid components. The present invention is particularly useful in analyzing blood samples, which include both a fluid component (serum) and a solid component (various types of cells). In particular, the invention is directed to analyzing cellular components in a blood sample, either whole blood (which contains various types of blood cells) or a cell component fraction. The present invention may also be used to analyze cells obtained from a tissue sample that are separated from connective tissue and suspended in a biologically compatible liquid medium that does not destroy the cells. The present invention may also be used to analyze body fluid samples, platelet rich plasma samples, instrument control fluid samples, etc. The terms “cellular analyzer” and “cellular analysis” are intended to cover at least all of the components as described herein.

Generation of Raw Data

The body fluids and/or cellular components of body fluids may be subjected to various types of analytical techniques to generate data for analysis and display in accordance with the present invention. The most common techniques are Direct Current to measure the volume of the cell size, Radio Frequency to measure the opacity of the cell, fluorescence, and light scatter to measure the granularity of the cell.

System Architecture

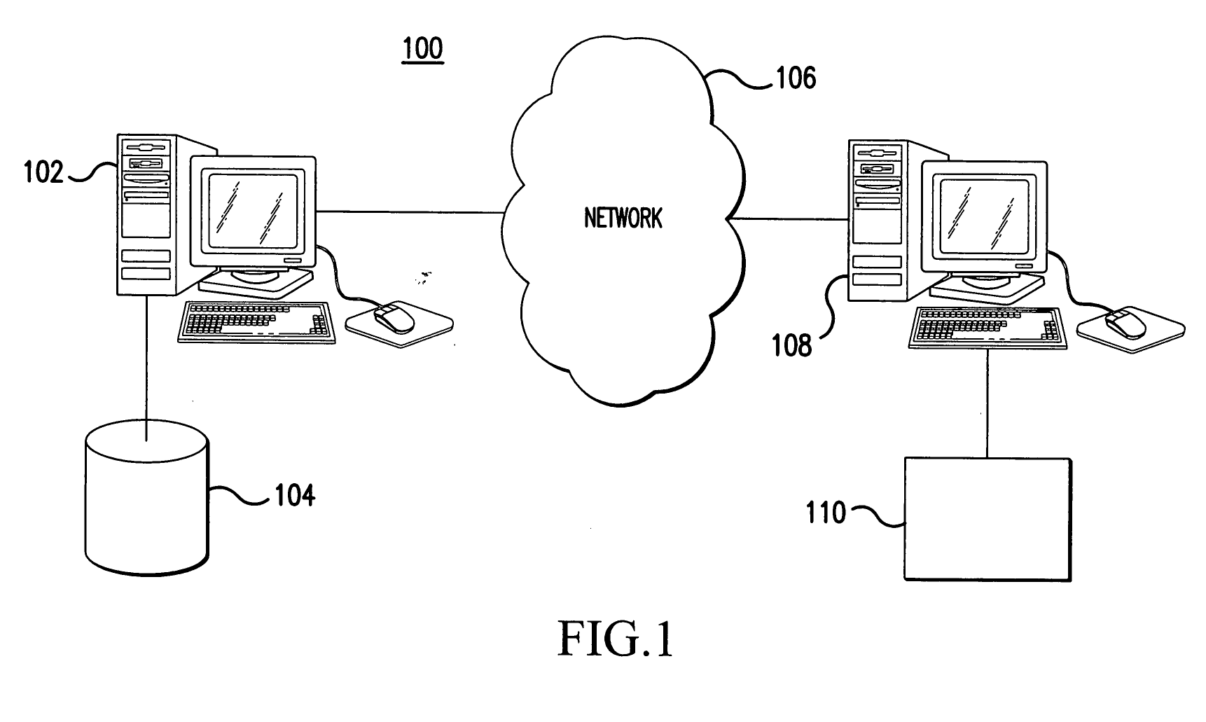

FIG. 1 is an exemplary diagram of a system environment 100 for implementing the principles of the present invention. The components of system 100 may be implemented through any suitable combinations of hardware, software, and/or firmware. As shown in FIG. 1, system 100 includes a user computer 102. User computer 102 may be communicably linked to a database 104. Alternatively, database 104 may reside directly on network 106 or the contents of database 104 may reside directly on computer 102 or server 108. Still alternatively, there may be no database within system 100 and any data that may be stored at database 104 may be stored at computer 102 or server 108.

System 100 may further include network 106 which may be implemented as the Internet, or any local or wide area network, either public or private. System 100 may further include server 108 and server 108 may be communicably linked to analyzer 110. Analyzer 110 may be implemented as Beckman Coulter hematology instruments, such as LH750 and LH500, etc., to generate the test result data.

It may be appreciated by one of ordinary skill in the art that while only one computer 102, database 104, network 106, server 108 and analyzer 110 are depicted, more than one of these types of devices may be implemented in the system consistent with the principles of some embodiments of the present invention. It may further be appreciated that each of these devices may reside in different locations within the system. For example, analyzer 110 may be directly communicably linked to computer 102, wherein computer 102 may receive data from analyzer 110 directly without operating over the network. It may still further be appreciated that features consistent with principles of the present invention may be implemented solely within computer 102 as a stand-alone unit where all of the data needed to perform the present invention may reside directly on computer 102 and wherein target sample data from analyzer 110 may be input by the user through an external device of computer 102.

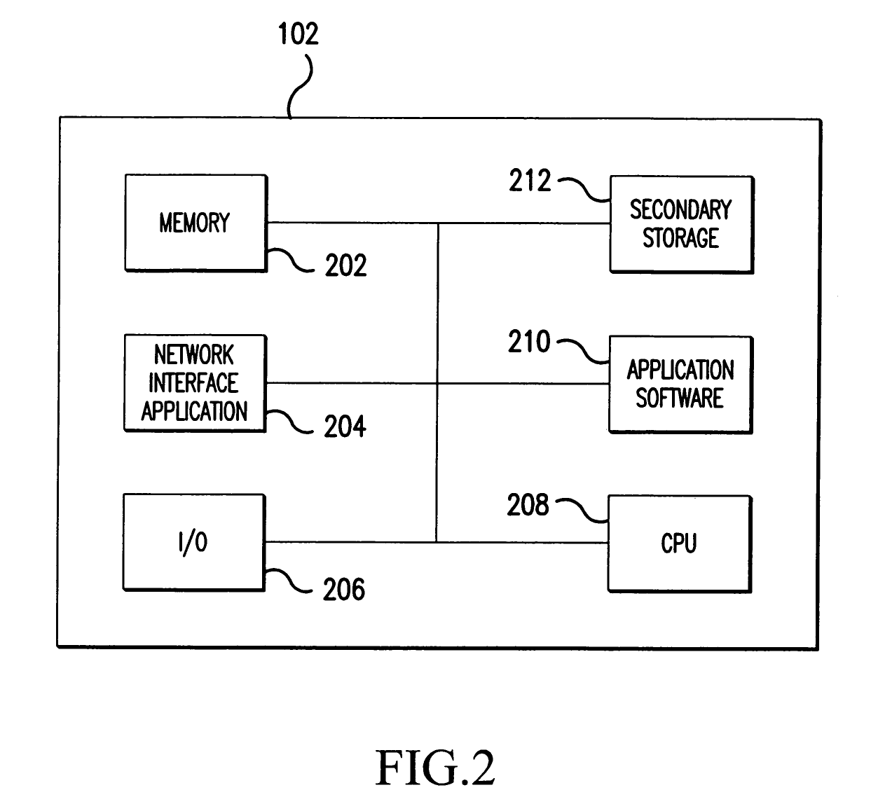

FIG. 2 depicts an exemplary block diagram of components included in computer 102. Computer 102 may be any type of computing device, such as a personal computer, workstation, or personal computing device, and may, for example, include memory 202, network interface application 204, input/output devices 206, central processing unit 208, application software 210, and secondary storage 212. Computer 102 may be communicably linked to database 104, server computer 108 and/or analyzer 110.

A user may access network 106 using the network interface application 204, and/or application software 210. Where network 106 may be implemented as the Internet, network interface application 204 may include a conventional browser including conventional browser applications available from Microsoft or Netscape. Application software 210 may include programming instructions for implementing features of the present invention as set forth herein. Application software 210 may include programming instructions for enabling a user to view and/or analyze test result data wherein target sample data is displayed together with template data. Application software 210 may further be implemented as Nevron Chart available from Nevron or MatLab available from Mathworks. Input/output devices 206 may include, for example, a keyboard, a mouse, a video cam, a display, a storage device, a printer, etc.

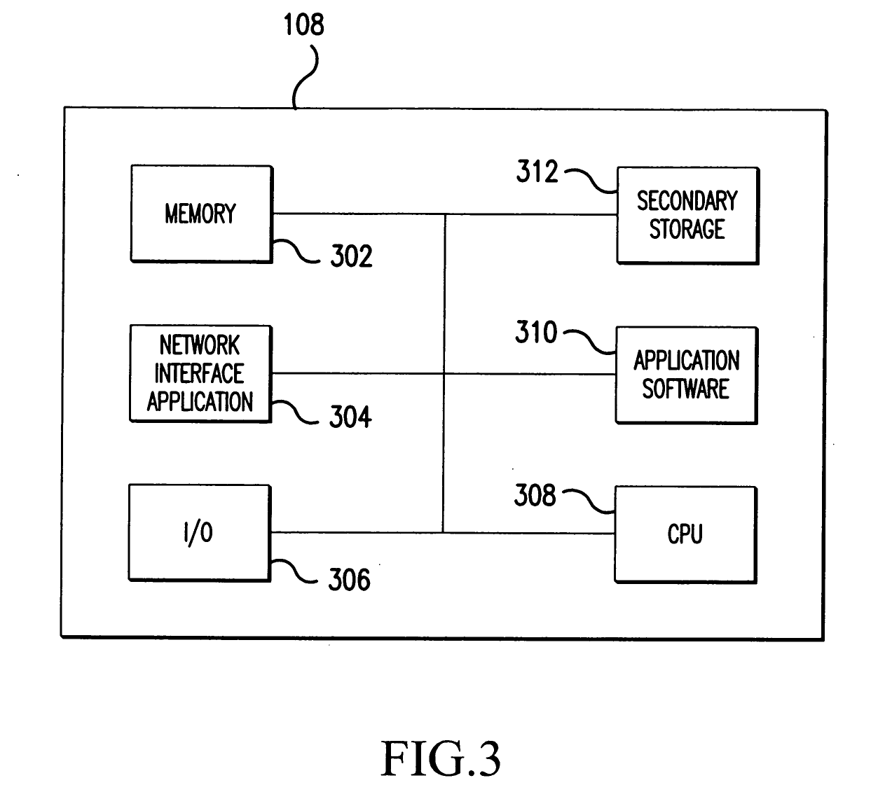

FIG. 3 depicts an exemplary block diagram of the components included in server computer 108. Server computer 108 may include memory 302, network interface application 304, input/output devices 306, central processing unit 308, application software 310, and secondary storage 312 consistent with the principles of some embodiments of the present invention. The components of server computer 108 may be implemented similarly with the components of computer 102.

Functionality

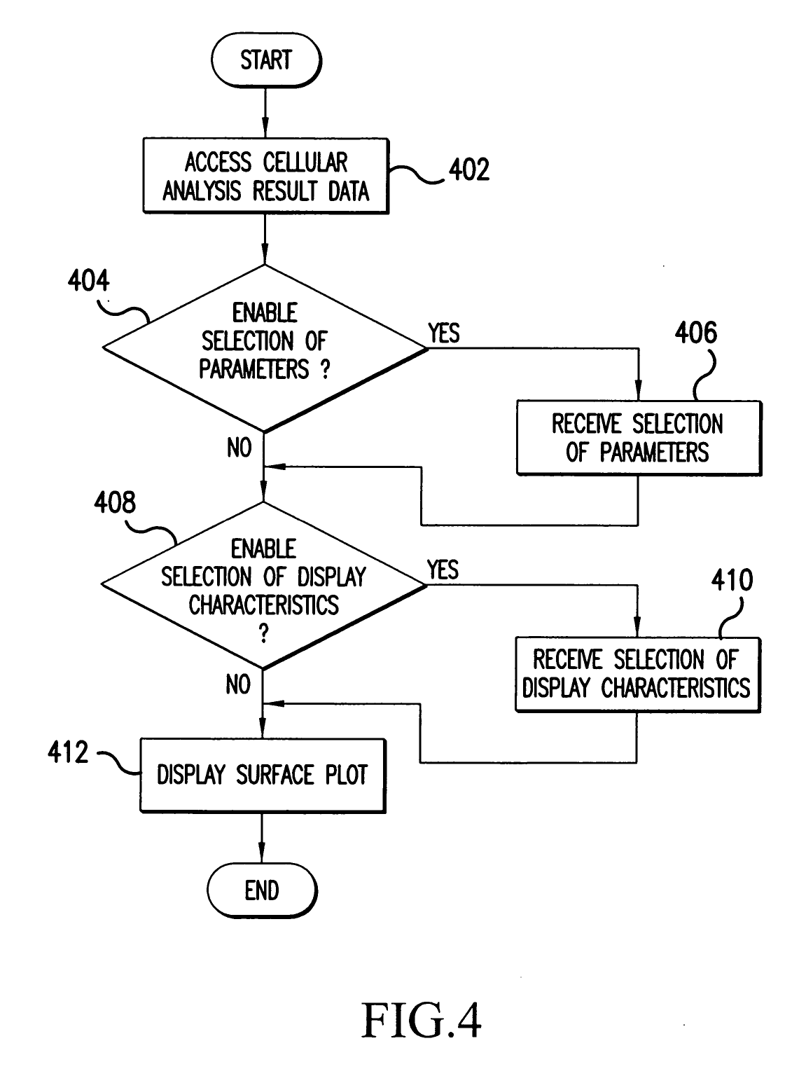

FIG. 4 depicts an exemplary flow diagram of the steps performed by computer 102, consistent with some embodiments of the present invention. As shown in FIG. 4, at the user's direction, the computer 102 may access the cellular analysis result data (Step 402). Computer 102 may then determine whether or not to enable selection by the user of parameters to identify what type of information to display (Step 404). If the system determines that the user may select the parameters, (Step 404, Yes), then using the user interface, the user may select the parameters identifying the data to display and the parameters are received by computer 102 (Step 406). For example, the user may select one or more types of cells to be included in the plot. If the system determines not to enable selection of parameters by the user (Step 404, No), the processing continues to Step 408. This determination of not enabling selection of parameters by the user may be because the parameters have already been predetermined or preset by computer 102.

Computer 102 may then determine whether or not to enable selection of display characteristics for each of the cell populations being displayed. If the system determines to enable selection of the display characteristics (Step 408, Yes), the user, using the user interface, may select the display characteristics and computer 102 then receives these selected display characteristics (Step 410). For example, the user may associate a color for each of the cell populations to be displayed so that, upon display of the surface plot, the user may easily identify each of the cell populations by their selected color. If the system determines not to enable selection of display characteristics by the user (Step 408, No), then processing proceeds to Step 412). The system may determine not to enable selection of the display characteristics if the display characteristics are already predetermined or preset. For example, by always associating the color red with the red blood cell population, then physicians may learn to automatically identify the red cell population by seeing the color red in a spike on the surface plot. Once the user enters all of the information, computer 102 displays a surface plot identifying the different cell populations with the difference display characteristics. The surface plot may be a three-dimensional surface plot. The displayed surface plot may be one or more predetermined views of a non-interactive surface plot. Alternatively, the displayed three-dimensional surface plot may be interactive wherein the user may zoom in and out of the image, spin around, over and/or under the image, etc.

It may be appreciated by one skilled in the art that the display characteristics may include gray scale, shading, texture, degree of opacity, etc.

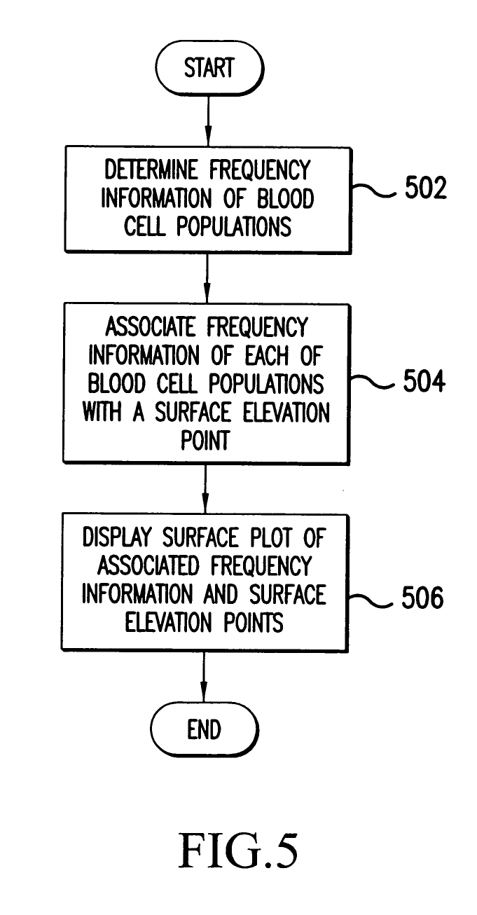

FIG. 5 depicts an exemplary flow diagram of the steps performed by computer 102 in generating and displaying the surface plot consistent with the principles of the present invention. As shown in FIG. 5, once the parameters are determined and the display characteristics are determined, computer 102 processes the cellular analysis result data and determines the frequency information of each of the determined cell populations (Step 502). Computer 102 then associates the frequency information of each of the cell populations with a surface elevation point (Step. 504). Finally, computer 102 displays a surface plot of the associated frequency information and surface elevation points (Step 506).

Displays

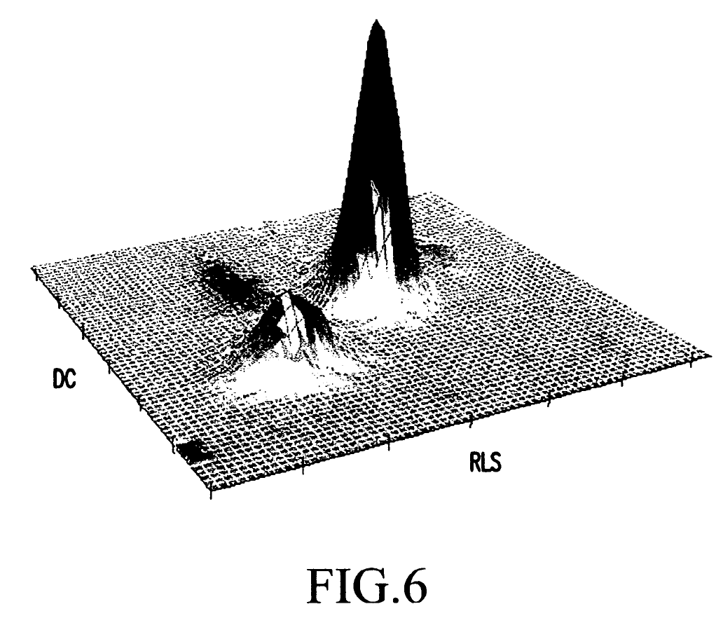

FIGS. 6-10 depict exemplary displays generated by system 100 and displayed to the user upon completion of the process set forth in FIGS. 4 and 5 consistent with the principles of some embodiments of the present invention. FIG. 6 depicts an exemplary color surface plot wherein different cell populations are identified by different display characteristics. Neutrophils are depicted in blue, lymphocytes are depicted in violet, monocytes are depicted in green, eosynophils are depicted in orange and red blood cells are depicted in red.

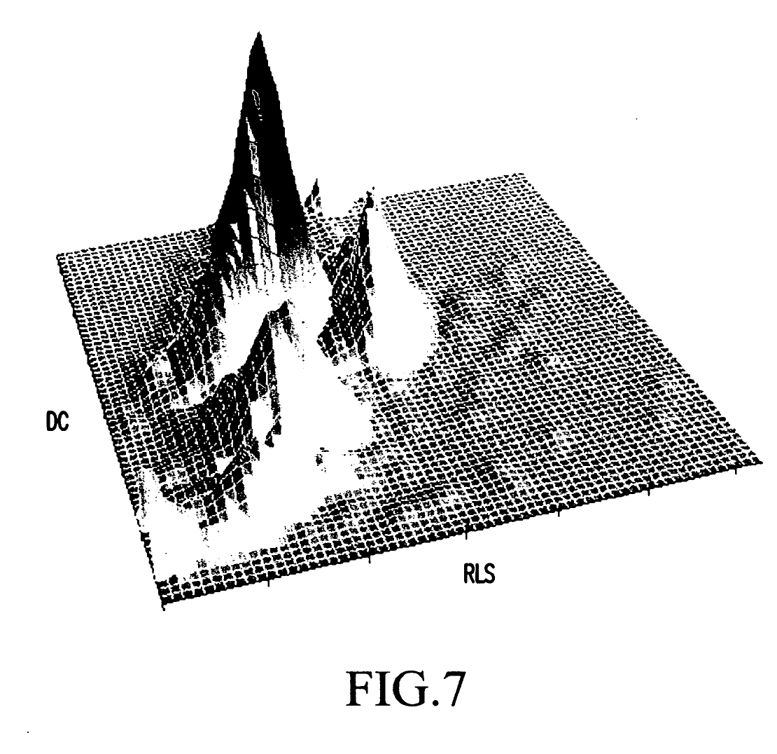

FIG. 7 depicts an exemplar color surface plot that shows a monoblast colored in red to give a clear indication of the abnormal cell population to the user.

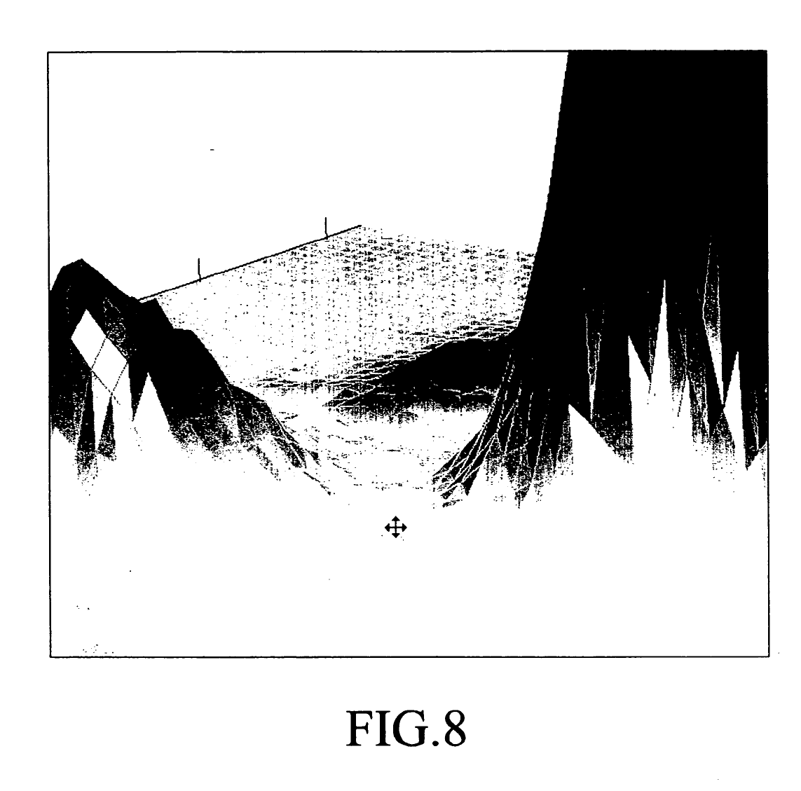

FIG. 8 depicts an interactive colored surface plot wherein the user has zoomed into the plot. The cross-hatch indicates the position of the user's mouse. By providing this functionality, the user is able to visually analyze the frequency information and to differentiate patterns in three dimensions.

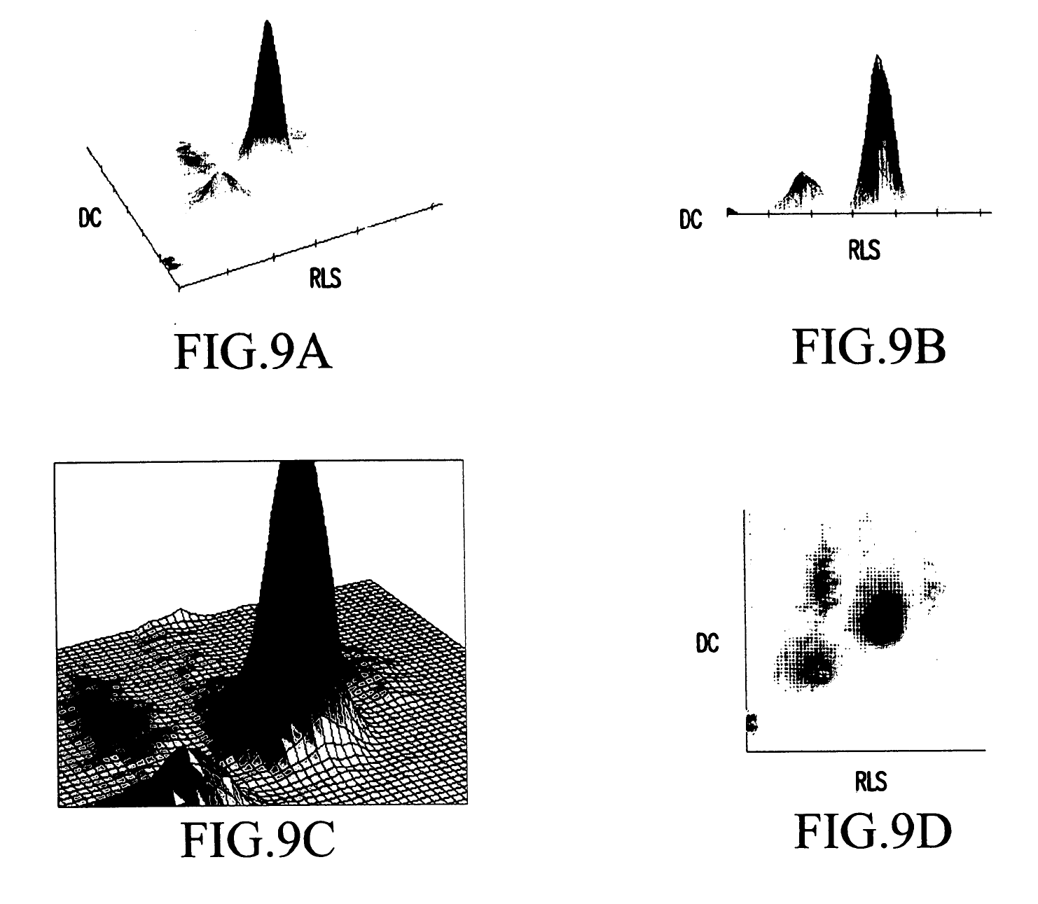

FIG. 9A-D depicts four predefined views depicting colored surface plots that may be presented to the user. Although these views may not be interactive, by providing several views to the user, the data may be more thoroughly analyzed.

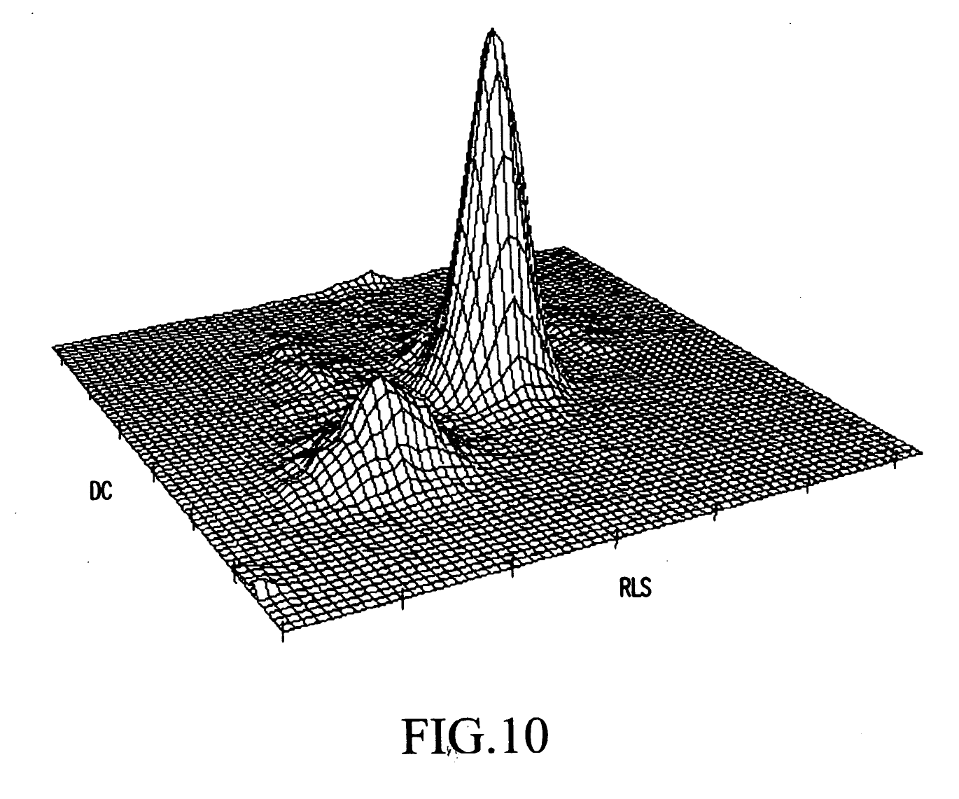

FIG. 10 depicts a black and white printout that depicts multiple cell populations.

Displaying the data using the features discussed herein may indicate to the user certain information regarding the data. For example, by inspecting the data displayed as discussed herein, the user may observe information regarding frequency distributions that may indicate a healthy or abnormal sample.

Conclusion

Modifications and adaptations of the present invention will be apparent to those skilled in art from consideration of the specification and practice of the invention disclosed herein. The foregoing description of an implementation of the invention has been presented for purpose of illustration and description. It is not exhaustive and does not limit the invention to the precise form disclosed. Modifications and variations are possible in light of the above teachings or may be acquired from the practicing of the invention. For example, the described implementation includes software, but systems and methods consistent with the present invention may be implemented as a combination of hardware and software or hardware alone.

Additionally, although aspects of the present invention are described for being stored in memory, one skilled in the art will appreciate that these aspects can also be stored on computer-readable media, such as secondary storage devices, for example, hard disks, or CD-ROM; the Internet or other propagation medium; or other forms of RAM or ROM.

We claim:

1. A method for displaying cellular analysis result data comprising:
accessing cellular analysis result data;
determining frequency information of a plurality of cell populations from the cellular analysis result data;
associating frequency information of each of the plurality of cell populations with a surface elevation point; and
displaying an interactive surface plot of the associated frequency information and the surface elevation points.

2. The method of claim 1, wherein the cellular analysis result data is from an automated hematology analyzer and is two-dimensional histogram data.

3. The method of claim 1, wherein displaying the interactive surface plot further includes displaying at least one of the cell populations in color.

4. The method of claim 3, wherein the at least one cell population displayed in color is colored with a color gradient representing identification of a specific cell population.

5. The method of claim 1, wherein the interactive surface plot is three-dimensional enabling a user to at least one of rotate the plot, move in, around, above, or below the plot, zoom into the plot, and zoom out of the plot.

6. The method of claim 1, wherein the surface plot enables a user to visually analyze the frequency information and to differentiate patterns in three dimensions.

7. The method of claim 1, wherein frequency information of the displayed interactive surface plot indicates a hematology anomaly.

8. A system for displaying cellular analysis result data comprising:
a memory for storing instructions;
a processor for executing the instruction to perform the method of:
accessing cellular analysis result data;
determining frequency information of a plurality of cell populations from the cellular analysis result data;
associating frequency information of each of the plurality of cell populations with a surface elevation point; and
displaying an interactive surface plot of the associated frequency information and the surface elevation points.

9. The system of claim 8, wherein the cellular analysis result data is from an automated hematology analyzer and is two-dimensional histogram data.

10. The system of claim 8, wherein displaying the interactive surface plot further includes displaying at least one of the cell populations in color.

11. The system of claim 10, wherein the at least one cell population displayed in color is colored with a color gradient representing identification of a specific cell population.

12. The system of claim 8, wherein the interactive surface plot is three-dimensional enabling a user to at least one of rotate the plot, move in, around, above, or below the plot, zoom into the plot, and zoom out of the plot.

13. The system of claim 8, wherein the surface plot enables a user to visually analyze the frequency information and to differentiate patterns in three dimensions.

14. The system of claim 8, wherein frequency information of the displayed interactive surface plot indicates a hematology anomaly.

15. A computer-readable medium storing instructions, executed by a processor, for performing a method for displaying cellular analysis result data, the method comprising:
accessing cellular analysis result data;
determining frequency information of a plurality of cell populations from the cellular analysis result data;
associating frequency information of each of the plurality of cell populations with a surface elevation point; and
displaying an interactive surface plot of the associated frequency information and the surface elevation points.

16. The computer-readable medium of claim 15, wherein the cellular analysis result data is from an automated hematology analyzer and is two-dimensional histogram data.

17. The computer-readable medium of claim 15, wherein displaying the interactive surface plot further includes displaying at least one of the cell populations in color.

18. The computer-readable medium of claim 17, wherein the at least one cell population displayed in color is colored with a color gradient representing identification of a specific cell population.

19. The computer-readable medium of claim 15, wherein the interactive surface plot is three-dimensional enabling a user to at least one of rotate the plot, move in, around, above, or below the plot, zoom into the plot, and zoom out of the plot.

20. The computer-readable medium of claim 15, wherein the surface plot enables a user to visually analyze the frequency information and to differentiate patterns in three dimensions.

21. The computer-readable medium of claim 15, wherein frequency information of the displayed interactive surface plot indicates a hematology anomaly.
